(19) United States
(12) Patent Application Publication
(10) Pub. No.: US 2001/0031974 A1
(43) Pub. Date: Oct. 18, 2001

(54) Neural regeneration conduit

(76) Inventors: Theresa A. Hadlock, Arlington, MA (US); Cathryn A. Sundback, Harvard, MA (US)

Correspondence Address: JANIS K. FRASER, Fish & Richardson P.C., 225 Franklin Street, Boston, MA, 02110-2804, US

(21) Appl. No.: 09/774,397

(22) Filed: Jan. 31, 2001

Related U.S. Application Data
(60) Provisional application No. 60/179,201, filed on Jan. 31, 2000.

(51) Int. Cl. A61B 17/08

(52) U.S. Cl. 606/152

(57) ABSTRACT

A neural regeneration conduit employing spiral geometry is disclosed. The spiral geometry is produced by rolling a flat sheet into a cylinder. The conduit can contain a multiplicity of functional layers lining the lumen of the conduit, including a confluent layer of adherent Schwann cells. The conduit can produce a neurotrophic agent concentration gradient by virtue of neurotrophic agent-laden microspheres arranged in a nonuniform pattern and embedded in a polymer hydrogen layer lining the lumen of the conduit.

### CROSS-REFERENCE TO RELATED APPLICATIONS

This application claims priority from U.S. Provisional Application Ser. No. 60/179,201, filed Jan. 31, 2000.

### TECHNICAL FIELD

This invention relates to neurology, cell biology and implantable prostheses, and particularly to methods and devices for surgical repair of transected or crushed nerves.

### BACKGROUND OF THE INVENTION

Peripheral nerve defects have been repaired by means of surgically implanting autograft nerves and with various types of implanted prostheses. Hollow entubulation conduits, autologous materials, e.g., vein or muscle grafts, allograft nerves and combinations of these approaches have been attempted with limited success. Schwann cells in a nerve gap, delivery of neurotrophic agents and isolation of a local regenerating milieu have been implicated in peripheral nerve regeneration. However, practical devices and methods for efficiently combining these components are needed.

### SUMMARY OF THE INVENTION

We have developed a neural regeneration conduit that employs spiral geometry. This advantageously permits formation of a multiplicity of functional layers lining the lumen of the conduit, including a confluent layer (e.g., monolayer) of adherent Schwann cells, and formation of neurotrophic agent concentration gradients.

The invention features a nerve regeneration conduit. The conduit includes: a porous biocompatible support which includes an inner surface and an outer surface, with the support being in the form of a roll. The roll is such that its cross section approximates a spiral spanning from 8 to 40 rotations, with the outer surface of the support facing outward, relative to the origin of the spiral. Preferably, a single layer of the support has a thickness of 5 μm to 200 μm, and more preferably 10 μm to 100 μm. The support can contain a naturally occurring biological material, for example, small intestinal submucosa (SIS), vein-derived tissue or a cellular dermal material. Alternatively, the support can contain a synthetic polymer. Suitable synthetic polymers include polyhydroxyalkanoates, e.g., polyhydroxybutyric acid; polyesters, e.g., polyglycolic acid (PGA); copolymers of glycolic acid and lactic acid (PLGA); copolymers of lactic acid and ε-aminocaproic acid; polycaprolactones; polydesoxazon (PDS); copolymers of hydroxybutyric acid and hydroxyvaleric acid; polyesters of succinic acid; polylactic acid (PLA); cross-linked hyaluronic acid; poly(organo)phosphazenes; biodegradable polyurethanes; and PGA cross-linked to collagen. In some embodiments, the support is bioresorbable.

Preferred embodiments of the invention include a layer of cells, for example, Schwann cells, adhered to the inner surface of the support. The conduit can contain from 15,000 to 165,000 Schwann cells per millimeter of conduit length. In some embodiments it contains from 20,000 to 40,000 Schwann cells per millimeter of conduit length, e.g., approximately 30,000 Schwann cells per millimeter of conduit length. The conduit can include a layer of extracellular matrix material, e.g., fibronectin, collagen or laminin, on the support.

The conduit can include a polymer hydrogel layer adhered to a layer of cells on the support, or to the support itself. Preferably the thickness of the hydrogel layer is 5 μm to 120 μm, and preferably 10 μm to 50 μm, e.g., approximately 25 μm. Materials suitable for use in a polymer hydrogel layer include fibrin glues, Pluronics®, polyethylene glycol (PEG) hydrogels, agarose gels, PolyHEMA (poly 2-hydroxyethylmethacrylate) hydrogels, PHPMA (poly N-(2-hydroxypropyl) methacrylamide) hydrogels, collagen gels, Matrigel®, chitosan gels, gel mixtures (e.g., of collagen, laminin, fibronectin), alginate gels, and collagen-glycosaminoglycan gels.

Some embodiments of the invention include a multiplicity of microspheres between the rolled layers of the support, e.g., immobilized in the hydrogel layer. The hydrogel layer can contain microspheres, a neurotrophic agent, or both. The neurotrophic agent can be incorporated directly into the hydrogel layer or loaded into microspheres. Suitable microsphere diameters range from of 1 μm to 150 μm. The microspheres can be formed from a material containing a copolymer of lactic acid and glycolic acid, preferably having an average molecular weight of 25 kD to 130 kD. In such a copolymer, the lactic acid:glycolic acid ratio can range from approximately 50:50 to almost 100% polylactic acid. In some embodiments, the ratio is approximately 85:15. Other materials also can be used to form the microspheres, e.g., polyhydroxyalkanoates, e.g., polyhydroxybutyric acid; polyesters, e.g., polyglycolic acid (PGA); copolymers of lactic acid and ε-aminocaproic acid; polycaprolactones; polydesoxazon (PDS); copolymers of hydroxybutyric acid and hydroxyvaleric acid; polyesters of succinic acid; and cross-linked hyaluronic acid. The microspheres can be arranged in a pattern to facilitate creation of a neurotrophic agent concentration gradient. Such a gradient can be radial or axial. Examples of useful neurotrophic agents are FK506 (tacrolimus), αFGF (acidic fibroblast growth factor), βFGF (basic FGF), 4-methylcatechol, NGF (nerve growth factor), BDNF (brain derived neurotrophic factor), CNTF (ciliary neurotrophic factor), MNGF (motor nerve growth factor), NT-3 (neurotrophin-3), GDNF (glial cell line-derived neurotrophic factor), NT4/5 (neurotrophin4/5), CM101, inosine, spermine, spermidine, HSP-27 (heat shock protein-27), IGF-I (insulin-like growth factor), IGF-II (insulin-like growth factor 2), PDGF (platelet derived growth factor) including PDGF-BB and PDGF-AB, ARIA (acetylcholine receptor inducing activity), LIF (leukemia inhibitory factor), VIP (vasoactive intestinal peptide), GGF (glial growth factor), IL-1 (interleukin-1), and neurotrophic pyrimidine derivative MS-430. The hydrogel layer can contain two or more neurotrophic agents. Different neurotrophic agents can be loaded into separate batches of microspheres, or two or more neurotrophic agents can be loaded into a single batch of microspheres.

The invention also features a method of manufacturing a nerve regeneration conduit. The method includes providing a porous, biocompatible support having an inner surface and an outer surface; and forming the support into a roll such that a cross section of the roll approximates a spiral spanning from 8 to 40 rotations, with the outer surface of the support facing outward, relative to the origin of the spiral. In addition, the method can include one or more of the following: culturing a layer (e.g., a monolayer) of cells on the support before forming the support into the roll, depositing a hydrogel layer and/or a multiplicity of microspheres on the support before forming the support into a role, loading a neurotrophic agent into the microspheres, and arranging the microspheres in a nonuniform pattern to facilitate neurotrophic agent concentration gradient formation.

The invention also features a method of facilitating regeneration of a transected nerve across a nerve gap defined by a proximal end of the transected nerve and a distal end of the transected nerve. The method includes: coapting the proximal end of the transected nerve to a first end of the conduit, and coapting the distal end of the transected nerve to a second end of the conduit.

The invention also features a method of facilitating regeneration of a crushed nerve. The method includes: providing a porous biocompatible support having an inner surface and an outer surface; culturing a layer of neurological cells (e.g., Schwann cells) on the support; and rolling the support around the crushed nerve. The method also can include depositing a hydrogel layer on the support before rolling the support around the crushed nerve, or incorporating a neurotrophic agent (e.g., via a microsphere or directly) into the hydrogel.

As used herein, “neurotrophic agent” means neurotropin or neurotrophin, i.e., any molecule that promotes or directs the growth of (1) neurons or portions thereof (e.g., axons), or (2) nerve support cells such as glial cells (e.g., Schwann cells).

The details of one or more embodiments of the invention are set forth in the accompanying drawings and the description below. Other features, objects, and advantages of the invention will be apparent from the description and drawings, and from the claims. All publications and other documents cited herein are incorporated by reference.

### BRIEF DESCRIPTION OF THE DRAWINGS

FIG. 1A is a schematic cross sectional view of a partially-rolled nerve regeneration conduit.

FIG. 1B is a schematic cross sectional view of a portion of a multilayered sheet used to form the nerve regeneration conduit in FIG 1A.

FIG. 2A is a schematic top view onto the inside surface of an unrolled conduit of the invention.

FIG. 2B is a cross-sectional view of the unrolled conduit shown in FIG. 2A, taken at line A-A. FIG. 2C is an end view of the conduit shown in FIGS. 2A and 2B, partially rolled according to arrow B in FIGS. 2A and 2B.

Like reference symbols in the various drawings indicate like elements.

### DETAILED DESCRIPTION OF THE INVENTION

The invention exploits the considerable advantages of “rolled architecture” in neural regeneration conduit. In rolled architecture, axial channels are replaced by a single spiraling axial space. This provides several advantages, including one or more of the following: (1) increased surface area for adherence of neural regeneration-supporting cells inside the conduit and to guide regeneration of an injured nerve; (2) a polymer hydrogel layer that provides an aqueous milieu for cell migration and neurotrophic agent diffusion; and (3) neurotrophic agents loaded into microspheres lining the inside of the conduit; (4) non-uniform geographic arrangement of microspheres to create axial or radial concentration gradient(s) of a single neurotrophic agent or multiple neurotrophic agents; (5 ) creation of a spatial gap (to accommodate regenerating nerves) by a hydrogel/microsphere layer acting as a spacer, or spacers joined or contiguous with the support, along the inside of the conduit; (6) choice of conduit materials; and (7) ease of manufacturing.

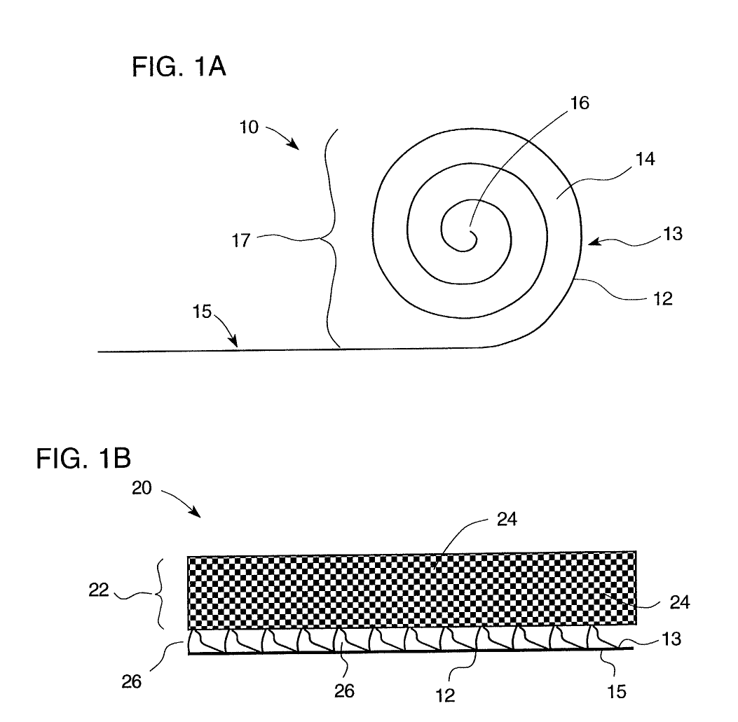

FIG. 1A is a cross sectional view of a partially-rolled nerve regeneration conduit 10. A porous support 12 has an outer surface 13 and an inner surface 15. An approximately spiral lumen 14 is created by rolling the support 12. Formation of a uniform space 14 between rolled layers of the support 12 is facilitated by a semi rigid hydrogel/microsphere layer (shown in FIG. 1B) adhered to the inner surface 15 of the support. The outer surface 13 faces outward with respect to the origin 16 of the spiral 17, and the inner surface 15 faces inward with respect to the origin 16 of the spiral 17. For ease of depiction, the schematic representation shows a partially-rolled conduit, whose spiral 17 lumen contains only approximately 3½ rotations. In preferred embodiments of the invention the spiral 17 contains from 8 to 40 rotations. The number of rotations will depend on various factors, including thickness of the support, thickness of the gap between support layers, and the desired outside diameter of the fully-rolled, cylindrical conduit. The conduit can be designed to have an outside diameter approximately matching the diameter of the nerve in which a gap is being bridged.

FIG. 1 B is a schematic, cross sectional view of a portion of a multilayered sheet 20 used to form the nerve regeneration conduit 10. A layer of Schwann cells 26 is adhered to the inner surface 15 of the porous support 12. Neurotrophin-laden microspheres 24 are embedded in a hydrogel layer 22.

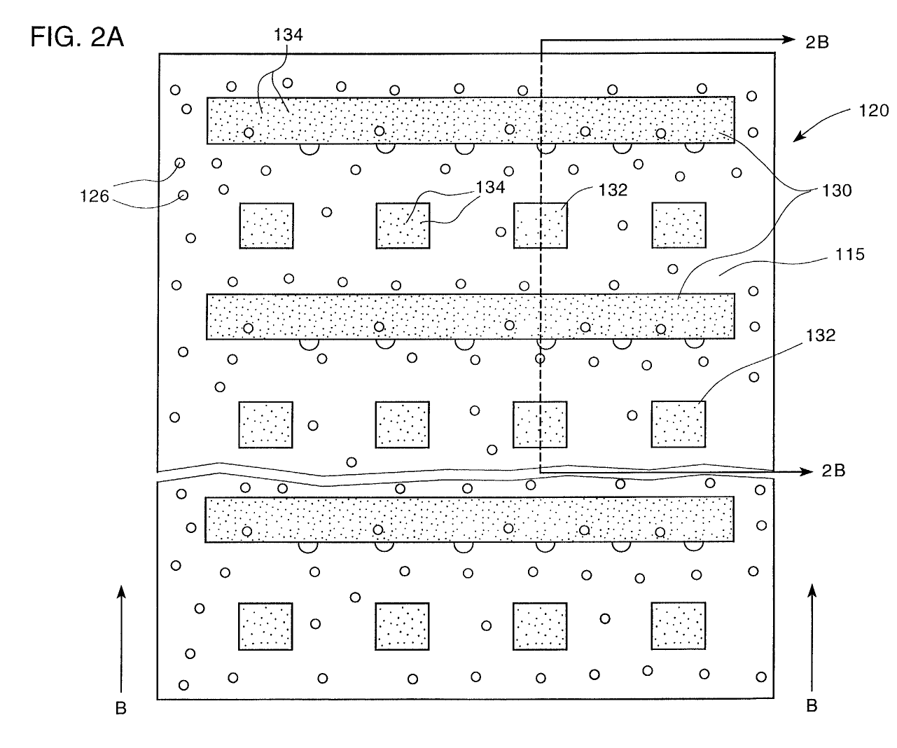

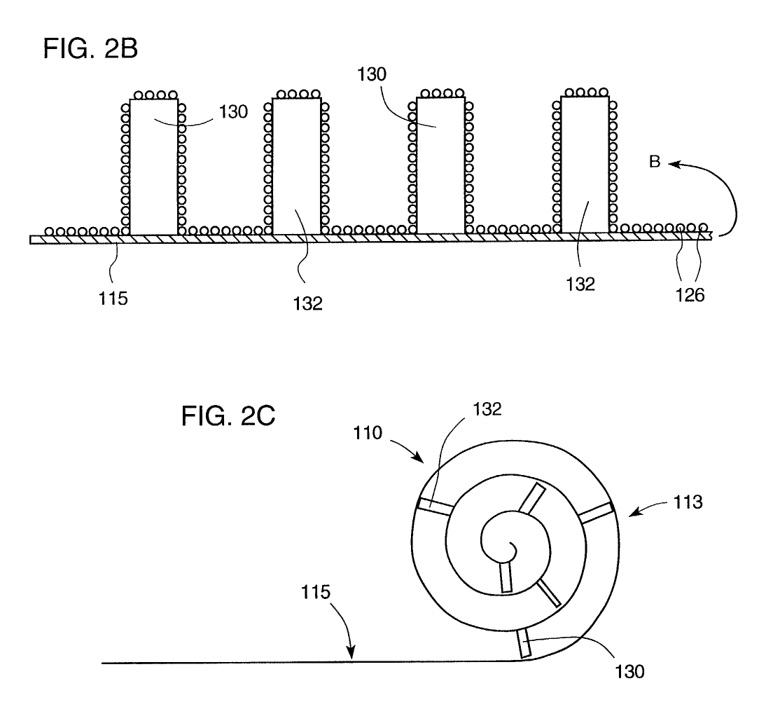

Referring to FIGS. 2A-2C, an alternative embodiment of a conduit is shown. FIG. 2A is a top view of an unrolled sheet 120, showing inside surface 115. Instead of a hydrogel layer providing spacing between layers of a roll, sheet 120 includes continuous spacers 130 and discontinuous spacers 132 (FIG. 2C). Of course, in other embodiments, a sheet can include either continuous or discontinuous spacers only. These spacers 130 and 132 and the rest of the sheet 120 can be formed from any castable foam material that is suitable for implantation, produced using microfabrication techniques, or formed using ink jet technology as described herein. Schwann cells 126 are adhered on inside surface 115. To form a rolled conduit 110, sheet 120 is rolled in direction B shown in FIGS. 2A and 2B. Rolled conduit 110 has outside surface 113.

Conduit 110 also includes an axial gradient of neurotrophin molecules 134 which are loaded into spacers 130 and 132. Such a gradient can be provided when the spacers and/or sheet is fabricated by ink jet technology. Alternatively, conduit 110 can be used in conjunction with microspheres and/or a hydrogel (not shown) that contain one or more neurotrophins, the microspheres being positioned between spacers 130 and 132.

### Conduit Support

There is considerable latitude in material used to form the conduit support 12. The material must be porous and biocompatible. In addition, it must have suppleness or ductility sufficient to permit rolling of the support into a compact, cylindrical structure, e.g., having a diameter approximately 0.5 to 3.0 mm, suitable for surgical implantation in the repair of transected or crushed nerves. Preferably, the support can be cut readily with surgical instruments, yet strong enough to anchor surgical sutures. In embodiments incorporating a layer of cells, the support should allow for adherence of cells. It is, however, important to note that cell adherence is not necessary for the operation of the invention. The thickness of the support 12 (single layer) can vary. Preferably it is from 5 to 200 μm, and more preferably, it is from 10 to 150 μm. Optimal thickness will depend on the material used to form the support 12, the size and anatomical location of the nerve to be repaired, and the length of the nerve gap (if any) to be bridged in the repair. After being formed by rolling, the cylindrical nerve conduit preferably displays at least some flexibility.

In some embodiments of the invention, the support 12 is formed partly or completely from a naturally occurring biological material. A suitable naturally occurring biological material is small intestinal submucosa (SIS). SIS is an a cellular collagen matrix that contains endogenous growth factors and other extracellular matrix components. Techniques for harvesting and handling SIS are known in the art. See, e.g., Lantz et al., J. Invest. Surg. 6:297-310 (1993). Other potentially useful natural, biological materials are vein tissue and a cellular material. In many embodiments of the invention, the support contains only non-immunogenic components. For example, SIS in not immunogenic. If immunogenic components are used, suitable immuno-suppressive therapy may be necessary. Such immunotherapy is known to those of skill in the art. See, e.g., Evans et al., Progress in Neurobiology 43:187-233, 1994.

In some embodiments of the invention, the support 12 is a thin sheet of synthetic polymer. Suitable synthetic polymers include polyhydroxyalkanoates, e.g., polyhydroxybutyric acid; polyesters, e.g., polyglycolic acid (PGA); copolymers of glycolic acid and lactic acid (PLGA); copolymers of lactic acid and ε-aminocaproic acid; polycaprolactones; polydesoxazon (PDS); copolymers of hydroxybutyric acid and hydroxyvaleric acid; polyesters of succinic acid; polylactic acid (PLA); cross-linked hyaluronic acid; poly(organo)phosphazenes; biodegradable polyurethanes; and PGA cross-linked to collagen. Poly(organo)phosphazene supports are described in Langone et al., Biomaterials 16:347-353, 1995. Polyurethane supports are described in Robinson et al., Microsurgery 12:412-419, 1991. The support can be bioresorbable, e.g., PLGA, or nonbioresorbable, e.g., SIS. In addition, the inclusion of an electrically conducting polymer (e.g., oxidized polypyrrole) in the conduit, in conjunction with electrical stimulation, can augment nerve repair. Such a strategy is described in Schmidt et al., Proc. Natl. Acad. Sci. USA 94:8948-8953, 1997.

The support and any structures contiguous with it (e.g., spacers) can be fabricated using any method known in the art. For example, the use of foam casting for generating prosthetic sheets with varying porosity can be adapted from processes described in Nam et al., Biomaterials 20:1783-1790, 1999; Nam et al., J. Biomed. Mat. Res. 47:8-17, 1999; and Schugens et al., J. Biomed. Mat. Res. 30:449-461, 1996. The porosity of biomaterials formed from casting can be controlled using differential concentrations of salts or sugars, CO₂ gas pressure, and other means known in the art. See, e.g., Lu et al., Biomaterials 21:1595-1605, 2000; Harris et al., J. Biomed. Mat. Res. 42:396-402, 1998; and Wake et al., Cell Transplantation 5:465 473, 1996. The pores in the foam should be large enough for exchange of gases and nutrients as necessary for cell maintenance, but small enough so that the surface of the support is impermeable to cells. A typical range suitable for a support of the invention is about 10-100 μm.

As an alternative to foam casting, microfabrication is a process that includes casting a polymer on top of a silicon wafer that has been etched. Most common polymers used in this process include polydimethylsiloxane (PDMS), which is non-biodegradable. However, microfabrication techniques can be adapted for biodegradable PLGA and the like, using a modification of the procedure described in Becker, Electrophoresis 21:12-26, 2000.

In some embodiments of the invention, it is desirable to deposit or impregnate the support with neurotrophins (e.g., a gradient of one or more neurotrophins) for facilitating axon migration and nerve regeneration in general. One means of accomplishing this task is to incorporate three-dimensional printing (3DP) ink jet printing technology into the manufacture of the support to produce a gradient of neurotrophins. General 3DP techniques as applied to medical devices is described in U.S. Pat. Nos. 5,490,962 and 5,869,170. If a gradient is not desired, a number of art-recognized methods can be used evenly distribute neurotropins throughout a support.

### Layer of Cells

In some embodiments of the invention, a monolayer of adherent cells 26 is cultured on the support 12 before it is rolled into a cylinder. Preferably, the cells 26 remain adhered to the support after the support is rolled into a cylinder for implantation. The cells 26 are employed for their ability to promote axonal extension of neurons in nerves. Schwann cells are particularly suitable, but any other adherent cell that promotes axonal extension can be employed. Alternatively, even if the Schwann cells do not adhere to the support, the cells can be encapsulated in the hydrogel described herein. Schwann cells encapsulated in hydrogels are described in Plant et al., Cell Transplantation 7:381-391, 1998; and Guenard et al., J. Neurosci. 12:3310-3320, 1992.

It is envisioned that a variety of cells can be included in the conduit to facilitate nerve regeneration. For example, the harvesting and use of olfactory ensheathing glial cells in nerve regeneration is described in Verdu et al., Neuroreport 10:1097-1101, 1999; and Ramon-Cueto et al., J. Neurosci. 18:3803-3815, 1998. In addition, neural stem cells, neural crest stem cells, or neuroepithelial cells can be harvested and optionally differentiated into neural support cells, such as described in Mujtaba et al., Dev. Biol. 200:1-15, 2000; Pardo et al., J. Neurosci. Res. 59:504-512, 2000; Mytilineou et al., Neurosci. Lett. 135:62-66, 1992; and Murphy et al., J. Neurosci. Res. 25:463-475, 1990. Alternatively, autologous bone marrow stromal cells can be differentiated into neural stem cells for use in a conduit. This conduit can then be grafted into the donor for nerve repair without the concern for graft rejection arising from implantation of allogenic or xenogenic cells. Isolation and differentiation of bone marrow stromal cells are described in Woodbury et al., J. Neurosci. Res. 61:364-370, 2000; and Sanchez-Ramos et al., Exp. Neurol. 164:247-256, 2000.

Optionally, the cells employed in the monolayer 26 are genetically engineered for one or more desirable traits, e.g., overexpression of a neurotrophic factor or axonal extension-promoting protein. Such cells need not be of glial cell origin, since the recombinant expression of neurotrophic factor in non-glial cells renders them suitable for use in the invention. In other words, recombinant expression converts originally non-nerve support cells into nerve support cells. Fibroblasts that express neurotrophins and are suitable for implantation are described in Nakahara et al., Cell Transplantation 5:191-204,1996 Examples of axonal extension-promoting proteins include NGF (Kaechi et al., J. Pharm. Exp. Ther. 272:1300-1304, 1995), FGF (Laird et al., Neuroscience 65:209 216, 1995), and GDNF (Frostic et al., Microsurgery 18:397-405, 1998). Other neurotrophins include FK506, 4-methylcatechol, BDNF, CNTF, MNGF, NT-3, NT-4/5, CM101, inosine, spermine, spermidine, HSP-27, IGF-I, IGF-II, PDGF (including PDGF-BB and PDGF-AB), IL-1, ARIA, LIF, VIP, GGF, and MS-430.

Production of a confluent layer of cells 26 on the support 12 can be accomplished readily through cell culture, using a mitogenic medium, and conventional animal cell culture techniques and equipment. Conventional cell culture techniques are known in the art and can found in standard references. See, e.g., Casella et al., Glia 17:327-338 (1996); Morrissey et al., J. Neuroscience 11:2433-2442 (1991).

In other embodiments, the cells can be grown on both the inside and outside surfaces of a support.

### Hydrogel Layer

Some embodiments of the invention include a polymer hydrogel layer 22 adhered to the support 12 or to a layer of cells 26 adhered to the support 12. The polymer hydrogel layer 22 can be any biocompatible, bioresorbable polymer gel that provides an aqueous milieu for cell migration and neurotrophic agent diffusion. The hydrogel can be natural or synthetic. The hydrogel layer 22 can have a thickness from 5 to 120 μm, preferably from 10 to 50 μm, e.g., approximately 20, 25 or 30 μm. Optimal hydrogel thickness depends on factors such as the diameter of the nerve being repaired and the number and diameter of microspheres 24 (if any) to be accommodated in the hydrogel layer 22. Exemplary materials for use in a polymer hydrogel layer 22 are fibrin glues, Pluronics®, polyethylene glycol (PEG) hydrogels, agarose gels, PolyHEMA (poly 2-hydroxyethylmethacrylate) hydrogels, PHPMA (poly N-(2-hydroxypropyl) methacrylamide) hydrogels, collagen gels, Matrigel®, chitosan gels, gel mixtures (e.g., of collagen, laminin, fibronectin), alginate gels, and collagen-glycosaminoglycan gels. The hydrogel layer 22 can contain one or more neurotrophic agents or axon extension-promoting proteins. Such neurotrophic agents can be loaded directly into the hydrogel 22, loaded into microspheres 24, or incorporated into the support or spacers as described herein.

### Microspheres

Some embodiments of the invention include microspheres between the rolled layers of the support. The microspheres can be held in place by any suitable means. For example, the microspheres can be immobilized in the hydrogel layer. The microspheres can be “blank,” i.e., containing no active ingredient. Blank microspheres are can serve as spacers to aid in producing a desired and constant spacing between laminations of the support in the spiral. Microspheres 24 useful in the invention can have diameters of approximately 1 μm to 150 μm. Preferably, the microspheres are made of a semi rigid, biocompatible, bioresorbable polymeric material. A suitable polymeric material is a high molecular weight (approx. 130 kD) copolymer of lactic acid and glycolic acid (PLGA). PLGA is well tolerated in vivo, and its degradation time can be adjusted by altering the ratio of the two co-monomers.

Besides serving as spacers, microspheres can be loaded with one or more neurotrophic agents, or any other active ingredient, so that they serve as drug delivery vehicles. Effective use of PLGA as a drug delivery vehicle is known in the art. See, e.g., Langer, Ann. of Biomed. Eng. 23:101, 1995; and Lewis, “Controlled release of bioactive agents from lactide/glycolide polymers,” in Chasin and Langer (eds.), Biodegradable Polymers as Drug Delivery Systems, Marcel Dekker, New York (1995).

A particularly advantageous feature of the invention is that microspheres loaded with a neurotrophic agent can be arranged in a pattern so as to result in an axial or radial concentration gradient in the lumen of the nerve regeneration conduit. Moreover, when two or more neurotrophic agents are employed, the agents can be loaded into separate batches of microspheres, which can then be differently arranged to produce independent concentration gradients for each of the different neurotrophic agents. Effects of neurotrophin concentration gradients are known in the art. See, e.g., Goodman et al., Cell 72:77-98, 1993; and Zheng et al., J. Neurobiol. 42:212-219, 2000. Utilization of such concentration gradient effects is within ordinary skill in the art. In some embodiments of the invention designed to create a neurotrophic agent concentration gradient, the two ends of the conduit differ from each other with respect to one or more neurotrophic agents. Such conduits may require implantation across a nerve gap in only one of two possible orientations. To ensure implantation in the proper orientation, the two ends of the conduit can be rendered visually distinguishable by any suitable means, e.g., a non-toxic dye marking on the conduit itself, or markings on a sterile wrapper or container.

### Surgical Procedures

Surgical procedures known in the art can be employed when using a nerve regeneration conduit of the invention to repair transected peripheral nerves. Suitable surgical procedures are described, for example, in Hadlock et al., Archives of Otolaryngology—Head & Neck Surgery 124:1081-1086, 1998; WO 99/11181; U.S. Pat. No. 5,925,053; WO 88/06871; Wang et al., Microsurgery 14:608-618, 1993; and Mackinnon et al., Plast. Reconst. Surg. 85:419-424, 1990.

### EXAMPLE

Schwann cells were isolated from neonatal Fisher rats. Small intestinal submucosa (SIS) was harvested from adult Fisher rats for use as a support material in a nerve regeneration conduit. The SIS was cut into 7 mm by 8 cm pieces and pinned out. Schwann cells were plated onto the SIS sheets and cultured until they reached confluence. The strips were then rolled into a laminar structure and implanted across a 7 mm gap in the rat sciatic nerve (n=12). Control animals received SIS conduits without Schwann cells (n=11) or an autograft repair (n=12).

At both 6 and 10½ weeks, functional recovery through the Schwann cell-laden SIS conduits, measured by sciatic function index, exceeded that through the cell-free conduits, but compared favorably with autografts.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A nerve regeneration conduit comprising a porous biocompatible support comprising an inner surface and an outer surface, the support being in the form of a roll such that a cross section of the roll approximates a spiral spanning from 8 to 40 rotations, with the outer surface of the support facing outward, relative to the origin of the spiral.

2. The nerve regeneration conduit of claim 1, wherein the support has a thickness of 5 to 200 μm.

3. The nerve regeneration conduit of claim 1, wherein the support has a thickness of 10 to 100 μm.

4. The nerve regeneration conduit of claim 1, wherein the support comprises a biological material.

5. The nerve regeneration conduit of claim 4, wherein the biological material is small intestinal submucosa.

6. The nerve regeneration conduit of claim 1, wherein the support comprises a synthetic polymer.

7. The nerve regeneration conduit of claim 1, wherein the support is bioresorbable.

8. The nerve regeneration conduit of claim 6, wherein the synthetic polymer is selected from the group consisting of polyhydroxyalkanoates, e.g., polyhydroxybutyric acid; polyesters, e.g., polyglycolic acid (PGA); copolymers of glycolic acid and lactic acid (PLGA); copolymers of lactic acid and ε-aminocaproic acid; polycaprolactones; polydesoxazon (PDS); copolymers of hydroxybutyric acid and hydroxyvaleric acid; polyesters of succinic acid; polylactic acid (PLA); cross-linked hyaluronic acid; poly(organo)phosphazenes; biodegradable polyurethanes; and PGA cross-linked to collagen.

9. The nerve regeneration conduit of claim 1, further comprising a layer of cells adhered to the inner surface of the support.

10. The nerve regeneration conduit of claim 9, wherein the cells are Schwann cells or olfactory ensheathing glial cells.

11. The nerve regeneration conduit of claim 10, wherein the layer contains from 15,000 to 165,000 Schwann cells per millimeter of conduit length.

12. The nerve regeneration conduit of claim 11, wherein the layer contains from 20,000 to 40,000 Schwann cells per millimeter of conduit length.

13. The nerve regeneration conduit of claim 9, further comprising a layer of extracellular matrix material on the support.

14. The nerve regeneration conduit of claim 1, further comprising a hydrogel layer.

15. The nerve regeneration conduit of claim 14, wherein the hydrogel layer has a thickness of 5 to 120 μm.

16. The nerve regeneration conduit of claim 15, wherein the hydrogel layer has a thickness of 10 to 50 μm.

17. The nerve regeneration conduit of claim 14, wherein the hydrogel layer comprises a polymer selected from the group consisting of fibrin glues, Pluronics®, polyethylene glycol (PEG) hydrogels, agarose gels, PolyHEMA (poly 2-hydroxyethylmethacrylate) hydrogels, PHPMA (poly N-(2-hydroxypropyl) methacrylamide) hydrogels, collagen gels, Matrigel®, chitosan gels, gel mixtures (e.g., of collagen, laminin, fibronectin), alginate gels, and collagen-glycosaminoglycan gels.

18. The nerve regeneration conduit of claim 1, further comprising a multiplicity of microspheres.

19. The nerve regeneration conduit of claim 18, wherein the microspheres are immobilized in a hydrogel layer.

20. The nerve regeneration conduit of claim 14, wherein the hydrogel layer comprises a neurotrophic agent.

21. The nerve regeneration conduit of claim 18, wherein the microspheres comprise a neurotrophic agent.

22. The nerve regeneration conduit of claim 18, wherein the microspheres have a diameter of 1 to 150 μm.

23. The nerve regeneration conduit of claim 18, wherein the microspheres comprise a material selected from the group consisting of a polyhydroxyalkanoate, a polyester, a copolymer of glycolic acid and lactic acid (PLGA), a copolymer of lactic acid and ε-aminocaproic acid, a polycaprolactones, polydesoxazon (PDS), a copolymer of hydroxybutyric acid and hydroxyvaleric acid, a polyester of succinic acid; and crosslinked hyaluronic acid.

24. The nerve regeneration conduit of claim 23, wherein the microspheres comprise PLGA having an average molecular weight of 25 kD to 130 kD.

25. The nerve regeneration conduit of claim 24, wherein the lactic acid:glycolic acid ratio is approximately 85:15.

26. The nerve regeneration conduit of claim 18, wherein the microspheres are arranged in a pattern to facilitate creation of a neurotrophic agent concentration gradient.

27. The nerve regeneration conduit of claim 26, wherein the gradient is radial.

28. The nerve regeneration conduit of claim 26, wherein the gradient is axial.

29. The nerve regeneration conduit of claim 20 or 21, wherein the neurotrophic agent is selected from the group consisting of FK506, αFGF, βFGF, 4-methylcatechol, NGF, BDNF, CNTF, MNGF, NT-3, GDNF, NT-4/5, CM101, inosine, spermine, spermidine, HSP-27, IGF-I, IGF-II, PDGF, ARIA, LIF, VIP, GGF, IL-1, and MS-430.

30. The nerve regeneration conduit of claim 20, wherein the hydrogel layer comprises two or more neurotrophic agents.

31. The nerve regeneration conduit of claim 21, wherein the microspheres comprise two or more neurotrophic agents.

32. The nerve regeneration conduit of claim 31, wherein the neurotrophic agents are in separate microspheres.

33. The nerve regeneration conduit of claim 31, wherein two or more neurotrophic agents are in a single microsphere.

34. A method of manufacturing a nerve regeneration conduit, the method comprising providing a porous biocompatible support comprising an inner surface and an outer surface; and forming the support into a roll such that a cross section of the roll approximates a spiral spanning from 8 to 40 rotations, with the outer surface of the support facing outward, relative to the origin of the spiral.

35. The method of claim 34, further comprising culturing a layer of cells on the support prior to forming the support into the roll.

36. The method of claim 34, further comprising depositing a hydrogel layer on the support before forming the support into a roll.

37. The method of claim 34, further comprising incorporating a multiplicity of microspheres into the conduit.

38. The method of claim 37, wherein the microspheres comprise a neurotrophic agent.

39. A method of facilitating regeneration of a transected nerve across a nerve gap defined by a proximal end of the transected nerve and a distal end of the transected nerve, the method comprising coapting the proximal end of the transected nerve to a first end of the conduit of claim 1, and coapting the distal end of the transected nerve to a second end of the conduit.

40. A method of facilitating regeneration of a crushed nerve, the method comprising providing a porous biocompatible support comprising an inner surface and an outer surface; culturing a layer of cells on the support; and rolling the support around the crushed nerve.

41. The method of claim 40, further comprising depositing a hydrogel layer on the support before rolling the support around the crushed nerve.

42. The method of claim 40, further comprising incorporating a multiplicity of neurotrophic agent-laden microspheres into the conduit.

43. The nerve regenerating conduit of claim 14, wherein the hydrogel further comprises cells.

44. The nerve regenerating conduit of claim 1, wherein the support further comprises spacer members extending from the inner surface of the support.

45. The nerve regenerating conduit of claim 1, wherein the support is loaded with one or more neurotrophins.

46. The nerve regenerating conduit of claim 45, wherein the one or more neurotrophins are distributed in a gradient in the support.
